(19) United States
(12) Plant Patent Application Publication
(10) Pub. No.: US 2019/0297763 P1
(43) Pub. Date: Sep. 26, 2019

(54) Rhododendron plant named "MARIAN'

(50) ;

(71) Applicant: Akatsuka Garden Co., Ltd., Tsu City (JP)

(72) Inventor: Yukio Kurabayashi, Tsu City (JP)

(21) Appl. No.: 15/932,617

(22) Filed: Mar. 23, 2018

(51) Int. Cl. A01H 6/36

(52) U.S. Cl. PLT/240

(57) ABSTRACT

‘Marian’ is a new variety of rhododendron. This variety was produced by the hybridization of the ‘Stardust’ variety with the ‘Hagoromo’ variety in 2010. The plant may be used, e.g., as a flowering plant for outdoor cultivation, or as a potted plant. ‘Marian’ is visibly appealing, and produces flowers comprising a corolla that comprises vivid purple-pink and light purple-pink colors.

Plant name and variety denomination: Latin name of the genus and species of the plant claimed: Rhododendron L. The claimed plant is a hybridization of different species. The parental lines of the claimed plant resulted from the hybridization of different species. Common names of the claimed plant's species: Rhododendron, Azalea, Azaleodendron. Variety denomination: ‘Marian’.

### BACKGROUND OF THE INVENTION

Rhododendron is a genus of woody plants in the Ericaceae family, either evergreen or deciduous, and found mainly in Asia, although it is also widespread throughout the highlands of the Appalachian Mountains of North America. Most species have brightly colored flowers that bloom from late winter through to early summer.

Both species and hybrid rhododendrons (including azaleas) are used extensively as ornamental plants in landscaping in many parts of the world, including both temperate and subtemperate regions. Many species and cultivars are grown commercially for the nursery trade.

Rhododendrons are often valued in landscaping for their structure, size, flowers, and the fact that many of them are evergreen. Azaleas are frequently used around foundations and occasionally as hedges, and many larger-leafed rhododendrons lend themselves well to more informal plantings and woodland gardens, or as specimen plants.

### BRIEF SUMMARY OF THE INVENTION

‘Marian’ is a new variety of rhododendron (more specifically azalea, still more specifically azaleodendron) bred by pollinating variety ‘Stardust’ with pollen from variety ‘Hagoromo’ in 2010. The plant may be used, e.g., as a flowering plant for outdoor cultivation or as a potted plant.

‘Marian’ is visibly appealing, and produces flowers comprising a corolla that has spots and vivid purple-pink and light purple-pink colors.

‘Stardust’ rhododendron (unpatented) is a rhododendron variety that originated in Japan in 2001. ‘Stardust’ was produced by the hybridization of the ‘Wedding Bouquet’ (unpatented) variety with the ‘Purple Splendour’ (unpatented) variety. This variety has a small tree size in the shape of a dome. The flowers of this variety have a small diameter with a pinkish-purple color. There are about 15 pieces of blossom flower in the shape of a small dome.

‘Hagoromo’ rhododendron (unpatented) is a rhododendron variety that originated in Japan in 2004. ‘Hagoromo’ was produced by the hybridization of the ‘Taiyo’ variety with the ‘Kifujin’ variety. This variety has upright and branch growth. The flowers of this variety have a medium diameter with edge colored white and pinkish. There are about 15 pieces of flower blossoms that form a round shape.

‘Marian’ was asexually reproduced by tissue culture in Japan at Kamiogoso2080, Anocho Toshima, Tsu-shi, Mie, 514-2322. Specifically, propagation was carried out by taking a growing point from a lateral bud of a sprout and cultivating the growing point, without use of callus. Plants propagated from a ‘Marian’ plant by asexual or vegetative means are uniform and stable.

### BRIEF DESCRIPTION OF THE DRAWINGS

FIG. 1 is a photograph of a ‘Marian’ plant body.

FIG. 2 is a photograph depicting the full view of young ‘Marian’ plants being cultivated in a divided tray.

FIG. 3 is a photograph showing an enlarged view of a ‘Marian’ flower.

FIG. 4 is a photograph of flowers of comparative variety Mrs. Fujii.

FIGS. 5A-D are photographs showing measurements of various different parts of ‘Marian’ plants. The units shown are centimeters.

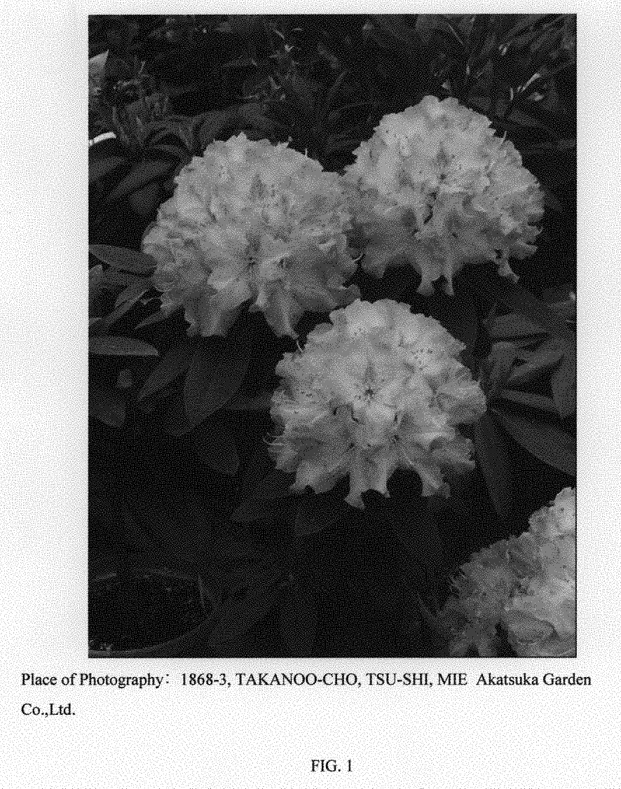

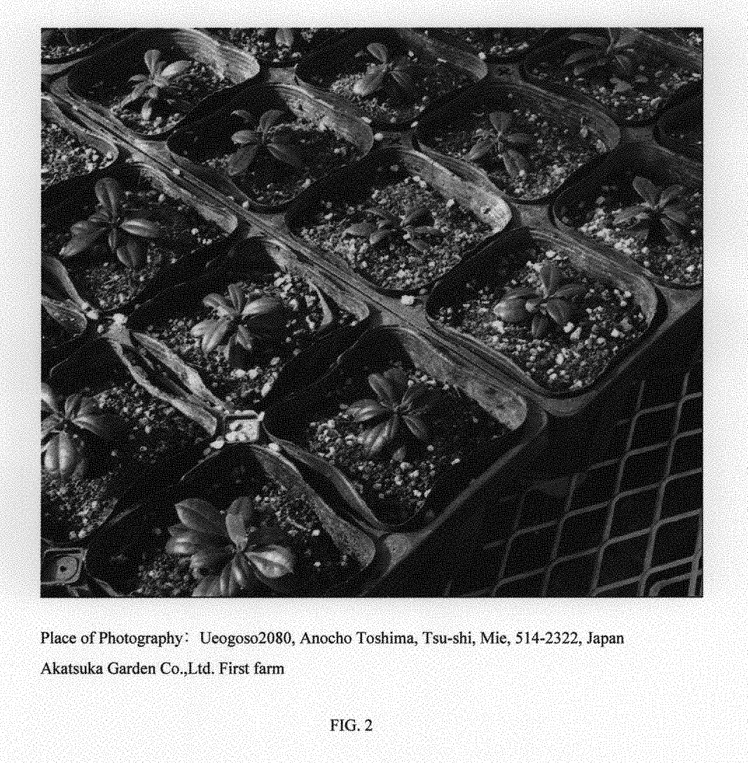

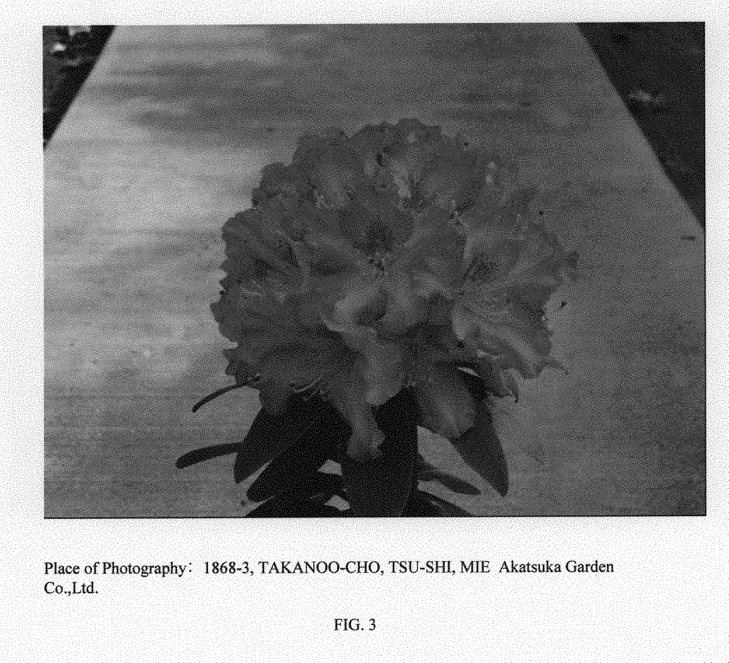

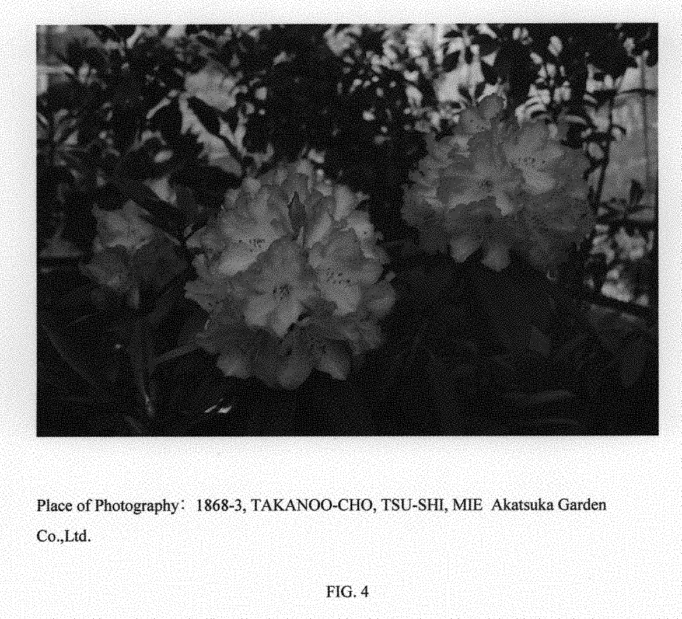

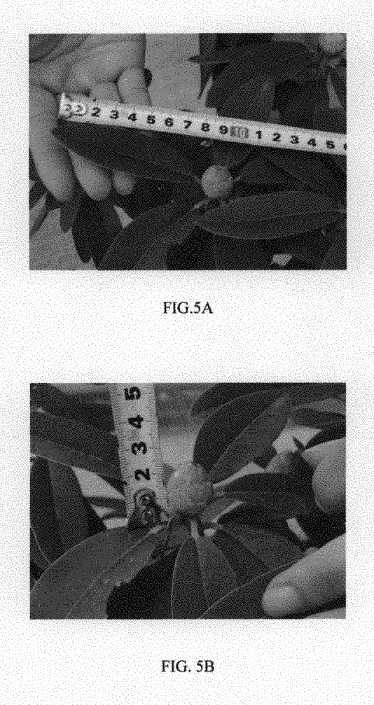

### DETAILED BOTANICAL DESCRIPTION OF THE PLANT

Variety: ‘Marian’

Species of the plant claimed: Rhododendron L. The claimed plant is a hybridization of different species. The parental lines of the claimed plant resulted from the hybridization of different species.

Common names of the claimed plant's species include: Rhododendron, Azalea, Azaleodendron

Characteristics of ‘Marian’ provided herein were observed when the plant was cultivated in the open with rain cover at Akatsuka Garden Co., Ltd., “first farm” having an address at Ueogoso2080, Anocho Toshima, Tsu-shi, Mie, 514-2322, Japan. Descriptive terms used in Tables 1, 2, and 3 herein are consistent with the Japanese Examination Standard for Rhododendron/Azalea (Rhododendron L.), TG/42/6 1995-10-20, pages 1-17, available at www.hinshu2.maff.go.jp/info/sinsakijun/kijun/1543.pdf, the entire contents of which are incorporated herein by reference.

Properties and characteristics of ‘Marian’ are described in Table 1.

TABLE 1
Properties and characteristics of ‘Marian’

| Charac- | | | Class Value |
|---|---|---|---|
| teristic | | | (Numeric Data) |
| No. | Description | State of Expression | of ‘Marian’** |
| 1 | Plant: persistence of | evergreen | 3 |
| | leaves | | |
| 24 | Flower: shape | open funnel-shaped | 2 |
| 25 | Flower: diameter | medium | 5 |
| 30 | Corolla lobe: number | Two | 2 |
| | of colors on upper side | | |
| | (excluding markings | | |
| | of the throat) | | |
| 31 | Corolla lobe: main | Japan color standard | Not applicable |
| | color on upper side | for horticultural plants | |
| | | (JHS) Color Chart No. | |
| | | 9504 (vivid purple- | |
| | | pink) | |
| 32 | Corolla lobe: | JHS Color Chart No. | Not applicable |
| | secondary color | 9502 (light purple- | |
| | on upper side | pink) | |
| 35 | Corolla lobe: | medium | 5 |
| | conspicuousness of | | |
| | markings of the throat | | |
| 36 | Corolla lobe: type of | spots not touching | 1 |
| | markings | each other | |
| 42 | Time of beginning of | late* | 7 |
| | flowering | | |

*Late April to early May, e.g., May 6th, when grown at Akatsuka Garden Co., Ltd., “first farm” having an address at Ueogoso2080, Anocho Toshima, Tsu-shi, Mie, 514-2322, Japan.
**Numerical Values in this column correspond to the “state” in the Table of Characteristics of the Japanese Examination Standard for Rhododendron/Azalea (Rhododendron L.), TG/42/6 1995-10-20, pages 1-17, available at www.hinshu2.maff.go.jp/info/sinsakijun/kijun/1543.pdf.

When grown in Tsu-shi, Mie, 514-2322, Japan (or in an areas with a similar latitude and climate) ‘Marian’ should be planted between early October to late March. In this region, flowering time is from late April to early May. Locations suitable for the growth of ‘Marian’ included mesic areas with good drainage and a soil pH of 5.0 to 5.5.

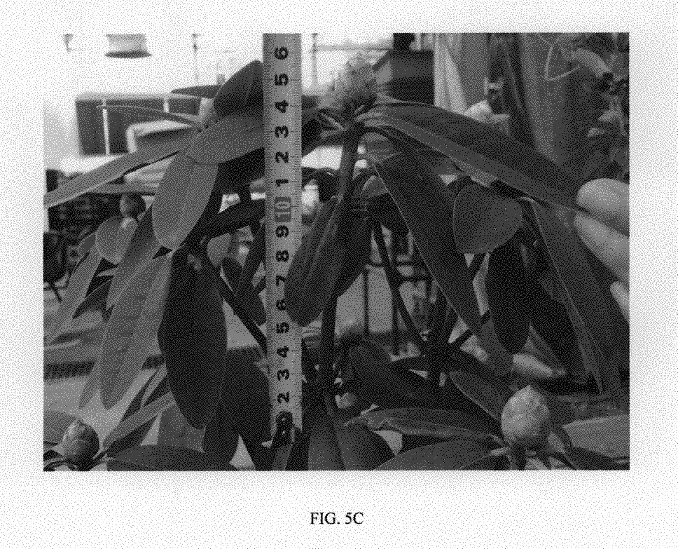

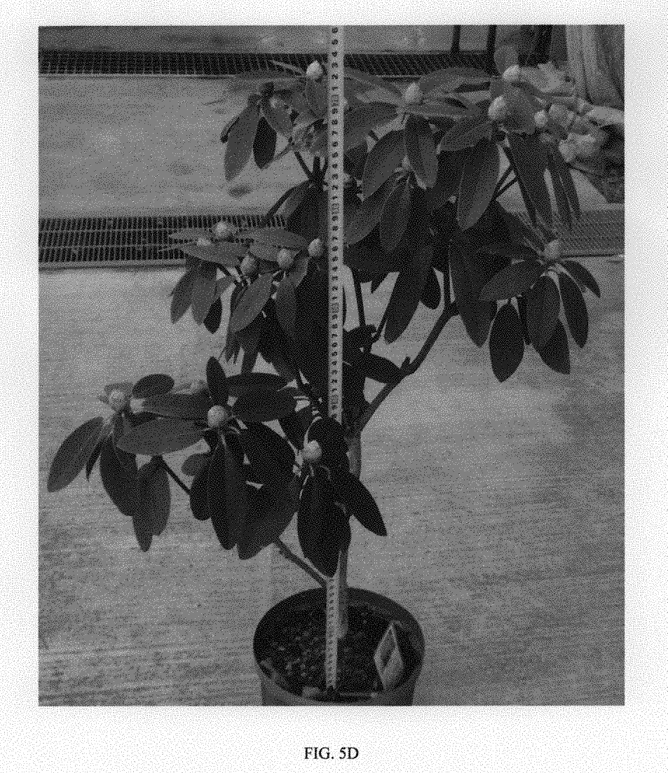

A variety that is similar to ‘Marian’ is ‘Mrs. Fujii’. Characteristics of ‘Marian’ and ‘Mrs. Fujii’ are compared in Table 2. Flowers of ‘Mrs. Fujii’ are shown in FIG. 5.

TABLE 2
Distinguishable Characteristics between ‘Marian’ and ‘Mrs. Fujii’

| Denomination | | State of | State of |
|---|---|---|---|
| of Similar | Description of | Expression in | Expression in |
| Variety | Characteristics | Similar Variety | Candidate Variety |
| Mrs. Fujii | Corolla lobe: | 9204 (vivid | 9504 (vivid |
| | main color on | purple-pink) | purple-pink) |
| | upper side | | |

A comparison between features of ‘Marian’ and its parental varieties is provided in Table 3.

TABLE 3
Description of Physical Differences between Parental Plants

| | Characteristic of | | |
|---|---|---|---|
| Variety | flower | Shape of tree, etc. | Origin |
| Claimed | Bright pink and | Upright. Small | First hybridized in |
| Variety: | medium diameter | size of tree and | 2010. Flowering |
| ‘Marian’ | flower with | branching is not | first observed in |
| | noticeable | violent. | 2013 |
| | brown spot. | | |
| | Around 25 pieces | | |
| | of blossom flower | | |
| | in the shape of ball. | | |
| Mother | Small diameter | Small size of tree | Original variety |
| Variety: | flowers, pinkish- | in the shape of | obtained by |
| ‘Stardust’ | purple. 15 pieces of | dome | hybridization of |
| | blossom flower in | | ‘WEDDING |
| | the shape of small | | BOUQUET’ |
| | dome. | | variety with |
| | | | ‘PURPLE |
| | | | SPLENDOUR’ |
| | | | variety in 2001. |
| Father | Medium diameter | Upright and | Original variety |
| Variety: | flower with edge | branched growth | obtained by |
| ‘Hagoromo’ | colored, white and | | hybridization of |
| | pinkish. Around 15 | | ‘TAIYO’ variety |
| | pieces of blossom | | with ‘KIFUJIN’. |
| | flower in a round | | variety in 2004 |
| | shape | | |

It will be understood that the average size of the plant may vary with location, season, nutrition, irrigation, etc.

What is claimed is:

1. A new and distinct Rhododendron L. plant named ‘Marian’ as illustrated and described.
